(19) United States
(12) Patent Application Publication
(10) Pub. No.: US 2006/0100255 A1
(43) Pub. Date: May 11, 2006

(54) Substituted indolealkanoic acids derivative and formulations containing same for use in the treatment of diabetic complications

(76) Inventor: Michael C. Van Zandt, Guilford, CT (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/522,642

(22) PCT Filed: Jul. 28, 2003
(86) PCT No.: PCT/US03/23527

§ 371 Date: Sep. 1, 2005

Related U.S. Application Data
(60) Provisional application No. 60/398,701, filed on Jul. 26, 2002.

(51) Int. Cl. A61K 31/427; C07D 417/02

(52) U.S. Cl. 514/367; 548/181

(57) ABSTRACT

Disclosed is a compound of the formula (I) its hydrates and/or pharmaceutically acceptable salts, which are useful in the treatment of chronic complications arising from diabetes mellitus. Also disclosed are pharmaceutical compositions containing the compounds and methods of treatment employing the compounds, as well as methods for their preparation.

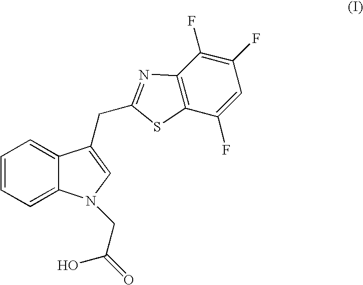

This application claims priority from U.S. Provisional Application Ser. No. 60/398,701, filed Jul. 26, 2002, the disclosure of which is incorporated herein in its entirety.

### BACKGROUND OF INVENTION

1. Field of the Invention

This invention relates to compounds and formulations for treating diabetic complications as well as processes for preparing the compositions and methods of treatment employing such formulations. More specifically, the invention relates to specific forms of indole acetic acids.

2. Description of the Related Art

The use of aldose reductase inhibitors (ARIs) for the treatment of diabetic complications is well known. The complications arise from elevated levels of glucose in tissues such as the nerve, kidney, retina and lens that enters the polyol pathway and is converted to sorbitol via aldose reductase. Because sorbitol does not easily cross cell membranes, it accumulates inside certain cells resulting in changes in osmotic pressure, alterations in the redox state of pyridine nucleotides (i.e. increased NADH/NAD⁺ ratio) and depleted intracellular levels of myoinositol. These biochemical changes, which have been linked to diabetic complications, can be controlled by inhibitors of aldose reductase.

The use of aldose reductase inhibitors for the treatment of diabetic complications has been extensively reviewed, see: (a) Textbook of Diabetes, 2nd ed.; Pickup, J. C. and Williams, G. (Eds.); Blackwell Science, Boston, Mass. 1997; (b) Aotsuka, T.; Abe, N.; Fukushima, K.; Ashizawa, N. and Yoshida, M., Bioorg. & Med. Chem. Letters, 1997, 7, 1677; and (c) T., Nagaki, Y.; Ishii, A.; Konishi, Y.; Yago, H; Seishi, S.; Okukado, N.; Okamoto, K., J. Med. Chem., 1997, 40, 684.

Aldose reductase inhibitors have been previously described in (a) U.S. Pat. No. 5,700,819; (b) U.S. Pat. No. 4,868,301; (c) U.S. Pat. No. 5,330,997; (d) U.S. Pat. No. 5,236,945; and U.S. Pat. No. 6,214,991. Although many aldose reductase inhibitors have been extensively developed, none have demonstrated sufficient efficacy in human clinical trials without significant undesirable side effects. Thus no aldose reductase inhibitors are currently available as approved therapeutic agents in the United States; and consequently, there is still a significant need for new, efficacious and safe medications for the treatment of diabetic complications.

### SUMMARY OF THE INVENTION

This invention provides crystal forms of {3-[(4,5,7-trifluoro-1,3-benzothiazol-2-yl)methyl]-1H-indol-1-yl}acetic acid, and pharmaceutical formulations containing this compound and/or its hydrates. The invention also provides methods of treating diabetic complications using the compound, and/or its hydrate(s), and/or its salts, to interact with aldose reductase. The compound {3-[(4,5,7-trifluoro-1,3-benzothiazol-2-yl)methyl]-1H-indol-1-yl}acetic acid has the following structure:

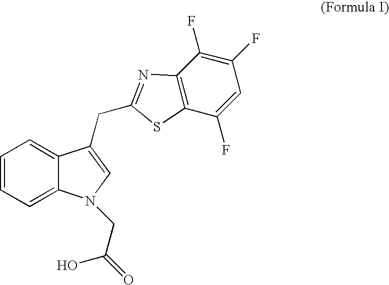

{3-[(4,5,7-trifluoro-1,3-benzothiazol-2-yl)methyl]-1H-indol-1-yl}acetic acid and/or its hydrate (hereinafter referred to as compounds of formula I) inhibit aldose reductase. Since aldose reductase is critical to the production of high levels of sorbitol in individuals with diabetes, inhibitors of aldose reductase are useful in preventing and/or treating various complications associated with diabetes. The compounds and compositions of the invention are therefore effective for the treatment of diabetic complications as a result of their ability to inhibit aldose reductase.

Thus, in another aspect, the invention provides methods for treating and/or preventing chronic complications associated with diabetes mellitus, including, for example, diabetic cataracts, retinopathy, nephropathy, and neuropathy.

In a related aspect, the invention provides methods of reducing sorbitol in tissues, specifically where the tissue is sciatic nerve, lens, retina, kidney cortex or kidney medulla, preferably such tissues in a diabetic patient.

Similarly, the invention provides methods of reducing fructose levels in tissues. Further, the invention provides methods of increasing myoinositol in tissues. The invention also encompasses methods of inhibiting the polyol-induced loss of nerve conduction velocity in the sciatic nerve, methods of reversing cataract formation, and methods of preventing cataract formation. Each of these methods is preferably directed to diabetic patients, more preferably human patients suffering from diabetes mellitus.

In still another aspect, the invention provides pharmaceutical compositions containing hydrates of {3-[(4,5,7-trifluoro-1,3-benzothiazol-2-yl)methyl]-1H-indol-1-yl}acetic acid and at least one pharmaceutically acceptable carrier, solvent, excipient or adjuvant.

In yet another aspect, the invention provides methods of making compounds of formula I and in particular, a method of making the monohydrate.

### BRIEF DESCRIPTION OF THE DRAWING

FIG. 1 is a flow chart showing the process for manufacturing the tablets and capsules of the invention.

### DETAILED DESCRIPTION OF THE INVENTION

As noted above, the invention provides a substituted indole alkanoic acid and/or its hydrates; these compounds are useful in treating and/or preventing complications associated with or arising from elevated levels of glucose in individuals, including humans and companion animals such as dogs, cats, and horses, preferably humans, suffering from diabetes mellitus.

The compounds and compositions of the invention I may be administered orally, topically, parenterally, by inhalation or spray or rectally in dosage unit formulations containing conventional non-toxic pharmaceutically acceptable carriers, adjuvants and vehicles. Preferably, the pharmaceutical compositions containing compounds and/or hydrates of Formula I may be in a form suitable for oral use, for example, as tablets, pills, troches, lozenges, aqueous or oily suspensions, dispersible powders or granules, emulsion, hard or soft capsules, or syrups or elixirs. A preferred route of administration is oral administration, more preferably oral administration of a composition that is a tablet or capsule. In these compositions, a compound or hydrate of Formula I may be present in association with one or more non-toxic pharmaceutically acceptable carriers and/or diluents and/or adjuvants and if desired other active ingredients.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example, lecithin, or condensation products of an, alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin, or cetyl alcohol. Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

Dosage levels on the order of from about 0.01 mg to about 100 mg, preferably to about 75 mg, more preferably to about 25 mg, of a compound or hydrate of Formula I, i.e., the active ingredient, per kilogram of body weight per day are useful in the treatment of the above-indicated conditions. More preferably, dosage levels are from about 0.025 mg to about 15 mg per kilogram of body weight per day. Even more preferably, dosage levels are from about 0.05 mg to about 10 mg per kilogram of body weight per day. Yet even more preferably, dosage levels are from about 0.05 mg to about 2.5 mg per kilogram of body weight per day. Particularly preferred dosage levels are from about 0.1 to about 0.5 mg per kilogram of body weight per day.

The amount of active ingredient that may be combined with the carrier materials to produce a single dosage form will vary depending upon the host treated and the particular mode of administration. Unit dosage forms will generally contain between from about 0.25 mg to about 1400 mg of the active ingredient. More preferably, unit dosage forms will generally contain between from about 0.5 mg to about 100 mg of the active ingredient. Even more preferably, unit dosage forms will generally contain between from about 1 mg to about 50 mg of the active ingredient. Still even more preferably, the unit dosage forms will generally contain between from about 1 mg to about 25 mg of the active ingredient. Particularly preferred dosage forms will contain from about 1 mg to about 15 mg of the active ingredient.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

The disclosures in this application of all articles and references, including patents, are incorporated herein by reference in their entirety.

The present invention is illustrated further by the following examples, which are not to be construed as limiting the invention in scope or spirit to the specific procedures and compounds described in them.

### EXAMPLE 1

### Preparation of 3-(4,5,7-Trifluorobenzothiazol-2-yl)methyl-indole-N-acetic Acid, monohydrate

### 2,3,5,6-Tetrafluoroacetanilide

A solution of 2,3,5,6-tetrofluoroaniline (200 g, 1.21 mol) in anhydrous pyridine (103 mL, 1.27 mol) was treated with acetic anhydride (120 mL, 1.27 mol) and heated to 120° C. for 2 h. After cooling to room temperature, the solution was poured into ice-cold water (500 mL). The resulting precipitate was filtered, dissolved in ethyl acetate, dried over MgSO₄, filtered, and concentrated. The solid material was washed with heptane (200 mL) and dried to give 2,3,5,6-tetrafluoroacetanilide as a white crystalline solid (206 g, 82%): mp 136-137° C.; R_{f} 0.48 (50% ethyl acetate in heptane); ¹H NMR (DMSO-d₆, 300 MHz) δ 10.10 (s, 1H), 7.87-7.74 (m, 1H), 2.09 (s, 3H). Anal. Calcd for C₈H₅F₄NO: C, 46.39; H, 2.43; N, 6.67. Found C, 46.35; H, 2.39; N, 6.68.

### 2,3,5,6-Tetrafluorothioacetanilide

A flame-dried, 4-necked 5,000 mL round-bottomed flask was charged with phosphorous pentasulfide (198 g, 0.45 mol) and diluted with anhydrous benzene (3,000 mL, 0.34 M). 2,3,5,6-tetrafluoroacetanilide (185 g, 0.89 mol) was added in one portion and the bright yellow suspension was heated to a gentle reflux for 3 h. The solution was cooled to 0° C. and filtered. The insoluble material was washed with ether (2×250 mL) and the combined filtrate was extracted with 10% aq. NaOH (750 mL, 500 mL). After cooling the aqueous layer to 0° C., it was carefully acidified with conc. HCl (pH 2-3). The precipitated product was collected by filtration and washed with water (500 mL). The yellow-orange material was dissolved in ethyl acetate (1,000 mL), dried over MgSO₄ and activated charcoal (3 g), filtered through a short pad of silica (50 g), and concentrated. The resulting solid was triturated with heptane (500 mL) and filtered to give 2,3,5,6-tetrafluorothioacetanilide (174.9 g, 88%): mp: 103-104° C.; R_{f} 0.67 (50% ethyl acetate in heptane); ¹H NMR (DMSO-d₆, 300 MHz) δ 11.20 (s, 1H), 8.00-7.88 (m, 1H), 2.66 (s, 3H). Anal. Calcd for C₈H₅F₄NS: C, 43.05; H, 2.26; N, 6.28. Found C, 43.10; H, 2.23; N, 6.19.

### 4,5,7-Trifluoro-2-methylbenzothiazole

A flame-dried 5,000 mL round-bottomed flask equipped with over-head stirrer was charged with sodium hydride (15.9 g, 0.66 mol) and diluted with anhydrous toluene (3,000 mL, 0.2 M). The suspension was cooled to 0° C., and treated with 2,3,5,6-tetrafluorothioacetanilide (134 g, 0.60 mol) in one portion. The solution was warmed to room temperature over 1 h, and then heated to a gentle reflux. After 30 min, dimethylformamide (400 mL) was carefully added and the mixture was stirred for an additional 2 h. The solution was cooled to 0° C. and added to ice-water (2,000 mL). The solution was extracted with ethyl acetate (1,500 mL) and washed with brine (1,000 mL). The organic layer was concentrated to dryness, diluted with heptane and successively washed with water (300 mL) and sat'd. aq. NaCl (1,000 mL). The organic layer was dried over MgSO₄, filtered, and concentrated to give 4,5,7-trifluoro-2-methylbenzothiazole (116.8 g, 96%) as a light brown solid: mp: 91-92° C.; R_{f} 0.56 (30% ethyl acetate in heptane); ¹H NMR (DMSO-d₆, 300 MHz) δ 7.76-7.67 (m, 1H), 2.87 (s, 3H); Anal. Calcd for C₈H₄F₃NS: C, 47.29; H, 1.98; N, 6.82; S, 15.78. Found C, 47.56; H, 2.07; N, 6.82; S, 15.59.

### 2-Amino-3,4,6-trifluorothiophenol Hydrochloride

A solution of 4,5,7-trifluoro-2-methylbenzothiazole (25.0 g, 123 mmol) in ethylene glycol (310 mL, 0.4 M) and 30% aq. NaOH (310 mL, 0.4 M) was degassed using a nitrogen stream then heated to a gentle reflux (125° C.) for 3 h. The solution was cooled to 0° C. and acidified to pH 3-4 using conc. HCl (approx. 200 mL). The solution was extracted with ether (750 mL) and washed with water (200 mL). The organic layer was dried over Na₂SO₄, filtered and treated with 2,2-di-tert-butyl-4-methylphenol (0.135 g, 0.5 mol %). After concentrating to dryness, the crude product was dissolved in anhydrous methanol (200 mL) and treated with an HCl solution in 1,4-dioxane (37 mL, 4 N, 148 mmol). The resulting mixture was concentrated to dryness, triturated with isopropylether (100 mL) and filtered to give 2-amino-3,4,6-trifluorothiophenol hydrochloride (19.3 g, 73%) as a light brown solid that was used without further purification. mp. 121-124 C; R_{f} 0.43 (30% ethyl acetate in heptane); Anal. Calcd for C₆H₅ClF₃NS: C, 33.42; H, 2.34; N, 6.50; S, 14.87. Found C, 33.45; H, 2.27; N, 6.48; S, 14.96.

### 3-cyanomethyl-indole-N-acetic acid, Ethyl Ester

Under an atmosphere of nitrogen, a solution of 3-indolyl acetonitrile (25.0 g, 160 mmol) in dry acetonitrile (530 mL, 0.3 M) was treated with sodium hydride (95%, 4.2 g, 168 mmol) and stirred for 30 min. Ethyl bromoacetate (21.3 mL, 192 mmol) was added in a dropwise manner over 10 min and the solution was stirred at room temperature for 16 h. After concentrating under reduced pressure, the resulting residue was dissolved in ethyl acetate and washed with sat'd. aq. NaCl. The organic extracts were dried over MgSO₄, filtered, and concentrated. The crude product was recrystallized from heptane and ethyl acetate to give the target compound as a white crystalline solid (19 g, 49%): mp 98-99° C.; R_{f} 0.29 (30% ethyl acetate in heptane); ¹H NMR (DMSO-d₆, 300 MHz) δ 7.59 (dd, J₁=7.8 Hz, J₂=0.6 Hz, 1H), 7.40 (dd, J₁=8.1 Hz, J₂=0.6 Hz, 1H), 7.36 (s, 1H), 7.18 (b t, J=7.2 Hz, 1H), 7.10 (b t, J=7.2 Hz, 1H), 5.12 (s, 2H), 4.14 (q, J=7.2 Hz, 2H), 4.06, (s, 2H), 1.20 (t, J=7.2 Hz, 3H); LRMS calcd for C₁₄H₁₄N₂O₂: 242.3; found 243.0 (M+1)⁺. Anal. Calcd for C₁₄H₁₄N₂O₂: C, 69.49; H, 5.82; N, 11.56. Found C, 69.39; H, 5.89; N, 11.59.

3-(4,5,7-trifluorobenzothiazol-2-yl)methyl-indole-N-acetic acid, Ethyl Ester: Under a nitrogen atmosphere, a solution of 3-acetonitrile-indole-N-acetic acid, ethyl ester (11.0 g, 45.4 mmol) in anhydrous ethanol (90 mL, 0.5 M) was treated with 2-amino-3,4,6-trifluorothiophenol hydrochloride (12.7 g, 59.0 mmol) and heated to a gentle reflux for 16 h. After cooling to room temperature, the solution was concentrated under reduced pressure, diluted with ethyl acetate and washed with 2N HCl and sat'd. aq. NaCl. The organic layer was dried over MgSO₄, filtered and concentrated. Purification by MPLC (10-50% ethyl acetate in heptane, 23 mL/min, 150 min) to give 3-(4,5,7-trifluorobenzothiazol-2-yl)methyl-indole-N-acetic acid, ethyl ester (6.0 g, 36%) as a white crystalline solid: mp 110-111° C.; R_{f} 0.41 (30% ethyl acetate in heptane); ¹H NMR (DMSO-d₆, 300 MHz) δ 7.74-7.66 (m, 1H), 7.54 (d, J=7.8 Hz, 1H), 7.46 (s, 1H), 7.40 (d, J=8.1 Hz, 1H), 7.15 (br t, J=6.9 Hz, 1H), 7.04 (br t, J=7.8 Hz, 1H), 5.14, s, 2H), 4.66 (s, 2H), 4.14 (q, J=7.2 Hz, 3H); LRMS calcd for C₂₀H₁₅F₃N₂O₂S: 404.4; found 405.0 (M+1)⁺. Anal. Calcd for C₂₀H₁₅F₃N₂O₂S; C, 59.40; H, 3.74; N, 6.93; S, 7.93. Found C, 59.52; H, 3.721 N, 6.92; S, 8.04.

### 3-(4,5,7-trifluorobenzothiazol-2yl) methyl-indole-N-acetic acid

A solution of give 3-(4,5,7-trifluorobenzothiazol-2-yl)methyl-indole-N-acetic acid, ethyl ester (5.91 g, 14.6 mmol) in 1,2-dimethoxyethane (73 mL, 0.2 M) was cooled to 0° C. and treated with aq. NaOH (1.25 N, 58 mL, 73.1 mmol) in a dropwise manner over 15 min. After the addition was complete, the solution was stirred for an additional 30 min, acidified to pH 3 with 2N HCl, and concentrated under reduced pressure. The residue was dissolved in ethyl acetate (200 mL) and washed with brine (30 mL). The organic extract was dried over Na₂SO₄, filtered, and concentrated. The resulting material was stirred as a suspension in heptane, filtered and dried to give 3-(4,5,7-trifluorobenzothiazol-2-yl)methyl-indole-N-acetic acid (5.38 g, 98%) as a pale yellow solid: mp 177-178° C.; R_{f} 0.44 (20% methanol in dichloromethane); ¹H NMR (DMSO-d₆, 300 MHz) δ 7.74-7.65 (m, 1H), 7.53 (d, J=7.5 Hz, 1H), 7.46 (s, 1H), 7.40 (d, J=8.1 Hz, 1H), 7.15 (b t, J=6.9 Hz, 1H), 7.03 (b t, J=7.2 Hz, 1H), 5.03 (s, 2H), 4.65 (s, 2H); LRMS calcd for C₁₈H₁₁F₃N₂O₂S: 376.4; found 375.0 (M−1)⁻. Anal. Calcd for C₁₈H₁₁F₃N₂O₂S: C, 57.44; H, 2.95; N, 7.44; S, 8.52. Found C, 57.58; H, 2.99; N, 7.38; S, 8.51.

### 3-(4,5,7-trifluorobenzothiazol-2yl) methyl-indole-N-acetic acid hydrate (“hydrate of formula I”)

The pale yellow 3-(4,5,7-trifluorobenzothiazol-2-yl)methyl-indole-N-acetic acid was recrystallization from acetonitrile and water to give 3-(4,5,7-trifluorobenzothiazol-2-yl)methyl-indole-N-acetic acid (4.53 g, 92%) as a white crystalline solid: mp 176-177° C.; R_{f} 0.44 (20% methanol in dichloromethane); ¹H NMR (DMSO-d₆, 300 Mhz) 7.74-7.65 (m, 1H), 7.53 (d, J=7.5 Hz, 1H), 7.46 (s, 1H), 7.40 (d, J=8.1 Hz, 1H), 7.15 (b t, J=6.9 Hz, 1H), 7.03 (b t, J=7.2 Hz, 1H), 5.03 (s, 2H), 4.65 (s, 2H); LRMS calcd for C₁₈H₁₁F₃N₂O₂S: 376.4; found 375.0 (M−1); Anal. Calcd for C₁₈H₁₃F₃N₂O₃S: C, 54.82; H, 3.32; N, 7.10; S, 8.13. Found C, 54.92; H, 3.32; N, 7.16; S, 8.23.

### EXAMPLE 2

The compound of Example 1 was tested for its potency, selectivity, and efficacy as an inhibitor of human aldose reductase. The potency or aldose reductase inhibiting effects were tested using methods similar to those described by Butera et al. in J. Med. Chem. 1989, 32, 757. Using this assay, the concentration required to inhibit human aldose reductase (hALR2) activity by 50% (IC50) was determined.

In a second assay, the same compound was tested for its ability to inhibit aldehyde reductase (hALR1), a structurally related enzyme. The test method employed was essentially the same as described by Ishii, et al., J. Med. Chem. 1996 39: 1924. Using this assay, the concentration required to inhibit human aldehyde reductase activity by 50% (IC50) was determined.

From these data, hALR1/hALR2 ratios were determined. Since high potency of test compounds as inhibitors of aldose reductase is desirable, low hALR2 IC50 values are sought. On the other hand, high potency of test compounds as inhibitors of aldehyde reductase is undesirable, and high hALR1 IC50s values are sought. Accordingly, the hALR1/hALR2 ratio is used to determine the selectivity of a test compound. The importance of this selectivity is described in Kotani, et al., J. Med. Chem. 40: 684, 1997.

The results of these tests are combined and presented in Table 1.

| | hALR2 | HALR1 | HALR1/ |
|---|---|---|---|
| Example # | (IC50) | (IC50) | hALR2 |
| 1 | 5 nM | 27,000 nM | 5,400 |
| Tolrestat | 13 nM | 1,940 nM | 149 |

The above results show the superior potency, selectivity and efficacy of the compound of Example 1. This compound is useful in the treatment of chronic complications arising from diabetes mellitus, such as diabetic cataracts, retinopathy and neuropathy. Accordingly, an aspect of the invention is treatment of such complications with the inventive compound; treatment includes both prevention and alleviation. The compound is useful in the treatment of, for example, diabetic cataracts, retinopathy, nephropathy and neuropathy.

### EXAMPLE 3

In a third set of experiments, the compound of Example 1 was assayed for its ability to normalize or reduce sorbitol accumulation in the sciatic nerve of streptozotocin-induced diabetic rats. The test methods employed to determine the efficacy are essentially those of Mylari, et al., J. Med. Chem. 34: 108, 1991.

{3-[(4,5,7-trifluoro-1,3-benzothiazol-2-yl)methyl]-1H-indol-1-yl}acetic acid monohydrate reduced sorbitol levels in tissues in a dose related manner. In a 15 day study of the rat sciatic nerve, {3-[(4,5,7-trifluoro-1,3-benzothiazol-2yl)methyl]-1H-indol-1-yl}acetic acid mono hydrate had a ED₅₀ of 1.3 mg/kg/day and a 100% inhibition of in sorbitol accumulation at a dose of 4.8 mg/kg/day. Similar beneficial changes in fructose (levels were reduced) and mysinositol (levels were increased) were observed as well.

### [(4,5,7-trifluoro-1,3-benzothiazol-2-yl)methyl]-1H-indol-1-yl}acetic acid monohydrate in reducing Tissue Sorbitol Levels in the STZ Induced Diabetic Rat

| | | % Sorbitol Reduction | | | | |
|---|---|---|---|---|---|---|
| Duration | In vivo dose | Sciatic | | | Kidney | Kidney |
| of Study | (mg/kg/day) | Nerve | Lens | Retina | Cortex | Medulla |
| 30 days | 5 | 100 | 88 | ND | ND | ND |
| 30 days | 10 | 100 | 90 | ND | ND | ND |
| 30 days | 25 | 100 | 95 | ND | ND | ND |
| 90 days | 5 | 100 | — | 89 | 33 | 36 |
| 90 days | 10 | 100 | — | 99 | 25 | 47 |
| 90 days | 25 | 100 | — | 96 | 45 | 58 |

ND = Not Determined.
“—” = cannot be accurately determined due to cataracts in untreated diabetes.

### EXAMPLE 5

3-[(4,5,7-trifluoro-1,3-benzothiazol-2-yl)methyl]-1H-indol-1-yl}acetic acid monohydrate is compared to the known se reductase inhibitors (ARI) zenarestat and zopolrestat in an 8-day STZ diabetic rat model. At a dose of 10 mg/kg/day, compounds of formula I are significantly more potent than either zenarestat or zopolrestat in lowering sorbitol levels in the sciatic nerve and lens.

| | In vivo dose | Sciatic Nerve | Lens sorbitol |
|---|---|---|---|
| ARI | mg/kg/day | sorbitol lowering | lowering |
| Hydrate of | 10 | 100% | 25-42% |
| formula 1 | | | |
| zenarestat | 10 | 52% | 0% |
| zopolrestat | 10 | 71% | 0% |

### EXAMPLE 6

In a 4-week study in STZ-diabetic rats, 3-[(4,5,7-trifluoro-1,3-benzothiazol-2-yl)methyl]-1H-indol-1-yl)acetic acid monohydrate, in a dose related fashion inhibited the polyol-induced loss of nerve conduction velocity (NCV) in the sciatic nerve. The decline was inhibited by 14%, 41%, and 79% at doses of 5, 10, and 25 mg/kg/day, respectively. This activity was confirmed in a second, longer study where 5, 10, and 25 mg/kg/day were administered to STZ-diabetic rats for 3 months. Data immediately follows.

| In vivo dose | | |
|---|---|---|
| mg/kg/day | Duration of Treatment | Improvement in NCV |
| 5 | 3 months | 50% |
| 10 | 3 months | 68% |
| 25 | 3 months | 105% |

### EXAMPLE 7

3-[(4,5,7-trifluoro-1,3-benzothiazol-2-yl)methyl]-1H-indol-1-yl}acetic acid monohydrate compares very favorably with the ability of zenarestat and zopolrestat to inhibit the decline in NCV. 3-[(4,5,7-trifluoro-1,3-benzothiazol-2-yl)methyl]-1H-indol-1-yl}acetic acid monohydrate has almost twice the activity of the known ARI compounds at comparable doses. Data immediately follows.

| | In vivo dose | Duration of | Duration of | Improvement |
|---|---|---|---|---|
| ARI | mg/kg/day | Study | Treatment | in NCV |
| Hydrate of | 25 | 4 weeks | 3 weeks | 79% |
| formula 1 | | | | |
| zenarestat | 32 | 2 weeks | 2 weeks | 48% |
| zopolrestat | 25 | 4 weeks | 4 weeks | 43% |

### Pharmaceutical Compositions

The following compositions are made essentially according to the process of Example 9.

Composition A
{3-[(4,5,7-trifluoro-1,3-benzothiazol-2-yl)methyl]-1H-indol-1-yl}acetic
acid monohydrate, 50 mg tablet

| Component | mg/tablet | weight % |
|---|---|---|
| Active compound * | 50 | 15.15 |
| Lactose fast flo | 248 | 75.15 |
| PVP | 16 | 4.85 |
| Purified water | qs | — |
| Croscarmellose sodium | 10 | 3.03 |
| Magnesium stearate | 6.0 | 1.82 |
| TOTAL | 330 mg | 100% |

* Active compound refers to (3-[(4,5,7-trifluoro-1,3-benzothiazol-2-yl)methyl]-1H-indol-1-yl}acetic acid monohydrate

Composition B
{3-[(4,5,7-trifluoro-1,3-benzothiazol-2-yl)methyl]-1H-indol-1-yl}acetic
acid monohydrate, 200 mg tablet

| Component | mg/tablet | weight % |
|---|---|---|
| Active compound * | 200 | 60.61 |
| Lactose fast flo | 98 | 29.69 |
| PVP | 16 | 4.85 |
| Purified water | qs | — |
| Croscarmellose sodium | 10 | 3.03 |
| Magnesium stearate | 6.0 | 1.82 |
| TOTAL | 330 mg | 100% |

* Active compound refers to {3-[(4,5,7-trifluoro-1,3-benzothiazol-2-yl)methyl]-1H-indol-1-yl}acetic acid monohydrate

Composition C
{3-[(4,5,7-trifluoro-1,3-benzothiazol-2-yl)methyl]-1H-indol-1-yl}acetic
acid monohydrate, 25 mg tablet

| Component | mg/tablet | weight % |
|---|---|---|
| Active compound * | 25 | 7.57 |
| Lactose fast flo | 273 | 82.73 |
| PVP | 16 | 4.85 |
| Purified water | qs | — |
| Croscarmellose sodium | 10 | 3.03 |
| Magnesium stearate | 6.0 | 1.82 |
| TOTAL | 330 mg | 100% |

* Active compound refers to (3-[(4,5,7-trifluoro-1,3-benzothiazol-2-yl)methyl]-1H-indol-1-yl}acetic acid monohydrate

Composition D
{3-[(4,5,7-trifluoro-1,3-benzothiazol-2-yl)methyl]-1H-indol-1-yl}acetic
acid monohydrate, 50 mg capsule

| Component | mg/tablet | % |
|---|---|---|
| Active compound * | 50 | 15.92 |
| Lactose fast flo | 248 | 78.98 |
| PVP | 16 | 5.10 |
| Purified water | qs | — |
| TOTAL | 314 mg | 100% |

* Active compound refers to {3-[(4,5,7-trifluoro-1,3-benzothiazol-2-yl)methyl]-1H-indol-1-yl}acetic acid monohydrate

Composition E
{3-[(4,5,7-trifluoro-1,3-benzothiazol-2-yl)methyl]-1H-indol-1-yl}acetic
acid monohydrate, 200 mg capsule

| Component | mg/tablet | % |
|---|---|---|
| Active Compound * | 200 | 63.69 |
| Lactose fast flo | 98 | 31.21 |
| PVP | 16 | 5.10 |
| Purified water | qs | — |
| TOTAL | 314 mg | 100% |

* Active compound refers to {3-[(4,5,7-trifluoro-1,3-benzothiazol-2-yl)methyl]-1H-indol-1-yl}acetic acid monohydrate

Composition F
{3-[(4,5,7-trifluoro-1,3-benzothiazol-2-yl)methyl]-1H-indol-1-yl}acetic
acid monohydrate, 25 mg capsule

| Component | mg/tablet | % |
|---|---|---|
| Active compound * | 25 | 7.96 |
| Lactose fast flo | 273 | 86.94 |
| PVP | 16 | 5.10 |
| Purified water | qs | — |
| TOTAL | 314 mg | 100% |

* Active compound refers to {3-[(4,5,7-trifluoro-1,3-benzothiazol-2-yl)methyl]-1H-indol-1-yl}acetic acid monohydrate

### EXAMPLE 9

Pharmaceutical Composition Manufacture

3-[(4,5,7-trifluoro-1,3-benzothiazol-2-yl)methyl]-1H-indol-1-yl)acetic acid monohydrate is stored at controlled room temperature (15-30° C.).

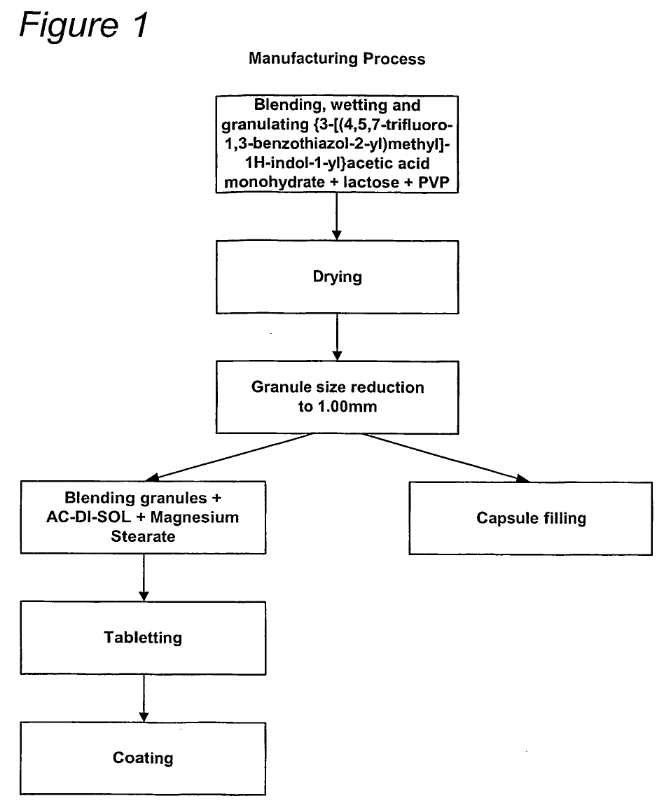

The following is a typical process description for a batch size of approximately 1,200 finished tablets having an amount of 200 mg3-[(4,5,7-trifluoro-1,3-benzothiazol-2-yl)methyl]-1H-indol-1-yl}acetic acid monohydrate per tablet. The process is generally shown in FIG. 1.

(1) The 3-[(4,5,7-trifluoro-1,3-benzothiazol-2-yl)methyl]-1H-indol-1-yl}acetic acid monohydrate, Lactose fast flo, and PVP are separately weighed into clean, tared and labeled polyethylene bags.

(2) Mix the 3-[(4,5,7-trifluoro-1,3-benzothiazol-2-yl)methyl]-1 h-indol-1-yl}acetic acid monohydrate, lactose and PVP in the Niro-fielder PP1 blender for 5 minutes at an impeller speed of 500 rpm, with no chopper.

(3) Purified water is added to the blender using a peristaltic pump while mixing at 500 rpm with the chopper (set at speed 2). The amount of water is approximately 60 mL. The addition of water is terminated when the granules are well formed.

(4) The wet granules are transferred from the blender to an Aeromatic fluid bed dryer and dried at 70° C. until the water content is between 1.0 and 2.5%.

(5) The dried granules are screened using a 1 mm screen on an Erweka AR400.

(6) Approximately 410 grams of granules are obtained. The weight of granules is corrected for water content. Required amounts of sodium croscarmellose and magnesium stearate are then calculated based on the corrected weight of the granules. The granules are then blended with the sodium croscarmellose in a Turbula mixer set at middle-speed setting for 6 minutes.

(7) Magnesium stearate is added to the blend and mixed at the slowest speed setting for 4 minutes.

(8) The blend is discharged into polyethylene bags.

(9) The blend is compressed using a Manesty F3 tablet press equipped with 10 mm normal concave punches. Compression produces tablet cores weighing about 330 mg (+5%).

(10) The thickness and hardness of the cores are determined and the cores are double bagged.

(11) Approximately 400 grams of tablet cores are obtained and quantities of Opadry and water are determined. The Opadry and water are mixed using a Heidolph magnetic stirrer for 30 minutes.

(12) An Aeromatic Strea 1 is prewarmed to 65° C. The spray gun is equipped with a 1 mm nozzle set at the minimum spray width, at the lowest spray position, and with the gun in a centralized position.

(13) The tablet cores are prewarmed in the Strea for 15 minutes with the fan setting at 10. The Opadry solution is placed in a Heidolph stirrer on a balance and the tube from the peristaltic pump placed into the solution.

(14) The initial settings for the spraying are adjusted to a temperature of 65° C., an atomising pressure of 1.2 bar, a fan capacity of 15, and a peristaltic pump setting of 7.

(15) The coating process is initiated and adjustments made to the fan capacity to ensure that the tablets are evenly coated. The spray rate is maintained between 4 and 5 grams/minute.

(16) After all of the solution has been sprayed, the tablets are dried for 10 minutes at about 65° C., fan capacity 10. The tablets are then allowed the tablets to come to room temperature and stored in double polyethylene bags.

The invention and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

What is claimed is:

1. Hydrates of a compound of the formula:

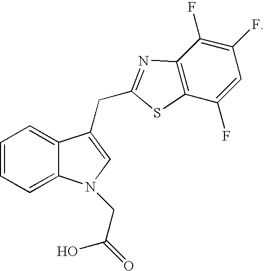

2. A compound that is {3-[(4,5,7-trifluoro-1,3-benzothiazol-2-yl)methyl]-1H-indol-1-yl}acetic acid monohydrate.

3. A pharmaceutical composition comprising an effective amount of a compound according to claim 1 or 2 and at least one pharmaceutically acceptable carrier, solvent, excipient or adjuvant.

4. A method of preventing or alleviating chronic complications arising from diabetes mellitus, which comprises administering to a mammal in need of such treatment an effective amount of a compound which is {3-[(4,5,7-trifluoro-1,3-benzothiazol-2-yl)methyl]-1H-indol-1-yl}acetic acid, or a pharmaceutically acceptable salt thereof, or a hydrate thereof.

5. A method according to claim 4, wherein the compound is {3-[(4,5,7-trifluoro-1,3-benzothiazol-2-yl)methyl]-1H-indol-1-yl}acetic acid monohydrate.

6. A method according to claim 5 wherein the mammal is a human.

7. A method according to claim 5 wherein the complications are selected from the group consisting of diabetic cataracts, retinopathy, nephropathy and neuropathy.

8. A method according to claim 5 wherein the complications are diabetic cataracts or retinopathy.

9. A method according to claim 5 wherein the complications are nephropathy or neuropathy.

10. A method of treatment according to claim 5 wherein the therapeutically effective amount for oral administration is about 0.01 mg to 100 mg/kilogram of body weight per day.

11. A method of treatment according to claim 10 wherein the therapeutically effective amount for oral administration is about 0.025 mg to 15 mg/kilogram of body weight per day.

12. A method of treatment according to claim 11 wherein the therapeutically effective amount for oral administration is about 0.05 mg to 10 mg/kilogram of body weight per day.

13. A method of treatment according to claim 10, wherein the therapeutically effective amount for oral administration is about 0.05 mg to 2.5 mg/kilogram of body weight per day.

14. A method according to claim 5, wherein the effective amount of the compound is contained within a unit dosage form containing about 1 to 10 mg of the compound.

15. A method according to claim 14, wherein the unit dosage form contains between about 0.5 mg to 100 mg of the compound.

16. A method according to claim 15 wherein the unit dosage form contains about 1 mg to 50 mg of the compound.

17. A method according to claim 16 wherein the unit dosage form contains about 1 mg to 15 mg of the compound.

18. A method of reducing sorbitol in tissues comprising administering from about 0.05 to 0.5 mg/kg/day of a compound which is {3-[(4,5,7-trifluoro-1,3-benzothiazol-2-yl)methyl]-1H-indol-1-yl}acetic acid or a salt or hydrate thereof.

19. A methods according to claim 24 wherein the tissue is sciatic nerve, lens, retina, kidney cortex or kidney medulla.

20. A method of reducing fructose levels in tissues comprising administering from about 0.05 to 0.5 mg/kg/day of a compound which is {3-[(4,5,7-trifluoro-1,3-benzothiazol-2-yl)methyl]-1H-indol-1-yl)acetic acid or a salt or hydrate thereof.

21. A method of increasing myoinositol in tissues comprising administering from about 0.05 to 0.5 mg/kg/day of a compound which is {3-[(4,5,7-trifluoro-1,3-benzothiazol-2-yl)methyl]-1H-indol-1-yl}acetic acid or a salt or hydrate thereof.

22. A method of inhibiting the polyol-induced loss of nerve conduction velocity in the sciatic nerve comprising administering from about 0.05 to 0.5 mg/kg/day of a compound which is {3-[(4,5,7-trifluoro-1,3-benzothiazol-2-yl)methyl]-1H-indol-1-yl}acetic acid or a salt or hydrate thereof.

23. A method of reversing cataract formation comprising administering from about 0.05 to 0.5 mg/kg/day of a compound which is {3-[(4,5,7-trifluoro-1,3-benzothiazol-2-yl)methyl]-1H-indol-1-yl}acetic acid or a salt or hydrate thereof.

24. A method of preventing cataract formation comprising administering from about 0.05 to 0.5 mg/kg/day of a compound which is {3-[(4,5,7-trifluoro-1,3-benzothiazol-2-yl)methyl]-1H-indol-1-yl}acetic acid or a salt or hydrate thereof.

25. A method according to claim 5 wherein the unit dosage form contains about 5 mg to 10 mg of the compound.

26. A pharmaceutical composition comprising {3-[(4,5,7-trifluoro-1,3-benzothiazol-2-yl)methyl]-1H-indol-1-yl}acetic acid monohydrate, lactose and polyvinylpyrrolidinone.

27. A pharmaceutical composition according to claim 26, wherein the composition is formulated into granules.

28. A pharmaceutical composition according to claim 27 wherein the granule size is less than 1.0 mm.

29. A pharmaceutical composition in tablet form comprising, by weight of the tablet, from about 5-75% of {3-[(4,5,7-trifluoro-1,3-benzothiazol-2-yl)methyl]-1H-indol-1-yl}acetic acid monohydrate, about 25-85% of lactose monohydrate, about 3-6% polyvinylpyrrolidinone, about 2-4% of croscarmellose sodium, and about 4-8% magnesium stearate.

30. A pharmaceutical composition in tablet form comprising about 50 mg of {3-[(4,5,7-trifluoro-1,3-benzothiazol-2-yl)methyl]-1H-indol-1-yl}acetic acid monohydrate, about 248 mg of lactose monohydrate, about 16 mg polyvinylpyrrolidinone, about 10 mg of croscarmellose sodium, and about 6 mg magnesium stearate.

31. A pharmaceutical composition in tablet form comprising about 200 mg of {3-[(4,5,7-trifluoro-1,3-benzothiazol-2-yl)methyl]-1H-indol-1-yl}acetic acid monohydrate, about 98 mg of lactose monohydrate, about 16 mg polyvinylpyrrolidinone, about 10 mg of croscarmellose sodium, and about 6 mg magnesium stearate.

32. A pharmaceutical composition in capsule form comprising about 200 mg of {3-[(4,5,7-trifluoro-1,3-benzothiazol-2-yl)methyl]-1H-indol-1-yl}acetic acid monohydrate, about 98 mg of lactose monohydrate, and about 16 mg polyvinylpyrrolidinone.

33. A pharmaceutical composition according to claim 32, wherein the capsule comprises granules of blended (3-[(4,5,7-trifluoro-1,3-benzothiazol-2-yl)methyl]-1H-indol-1-yl}acetic acid monohydrate, lactose monohydrate, and polyvinylpyrrolidinone.

34. A pharmaceutical composition according to claim 33, where the granules have an average size of about 1 mm.

35. A process for preparing a pharmaceutical composition according to any of claims 29-34, comprising forming granules of blended {3-[(4,5,7-trifluoro-1,3-benzothiazol-2-yl)methyl]-1H-indol-1-yl}acetic acid monohydrate, lactose monohydrate, and polyvinylpyrrolidinone, where the granules have an average size of about 1 mm.

36. A process for preparing a compound of claim 1, comprising forming a solution of {3-[(4,5,7-trifluoro-1,3-benzothiazol-2-yl)methyl]-1H-indol-1-yl}acetic acid in acetonitrile and water, and subsequently allowing crystals of the compound of claim 1 to form.

37. A process according to claim 36, wherein the solution is heated.

38. A pharmaceutical composition in tablet form comprising about 20-30 mg of {3-[(4,5,7-trifluoro-1,3-benzothiazol-2-yl)methyl]-1H-indol-1-yl}acetic acid monohydrate, about 270-280 mg of lactose monohydrate, about 10-20 mg polyvinylpyrrolidinone, about 5-15 mg of croscarmellose sodium, and about 3-10 mg of magnesium stearate.

39. A method normalizing sorbitol levels in tissues in a human patient, which comprises administering to a a patient in need of such treatment an effective amount of a compound according to claim 1.
